(19) United States
(12) Plant Patent Application Publication
(10) Pub. No.: US 2002/0129421 P1
(43) Pub. Date: Sep. 12, 2002

(54) DAHLIA PLANT NAMED 'HGD-161-7'

(75) Inventor: Johannes J.G. Sijm, Andijk (NL)

Correspondence Address: Foley & Lardner, 3000 K Street, N.W., Suite 500, Washington, DC, 20007-5109, US

(73) Assignee: HEM GENETICS BV

(21) Appl. No.: 09/801,680

(22) Filed: Mar. 9, 2001

(51) Int. Cl. A01H 5/00

(52) U.S. Cl. PLT/321

(57) ABSTRACT

A new and distinct cultivar of Dahlia plant named ‘HGD161-7’ characterized by its vigorous growth, shape of flowers, abundance of flowers, compact plants and fast response. The flowers have contrasting colors between ray and disc florets.

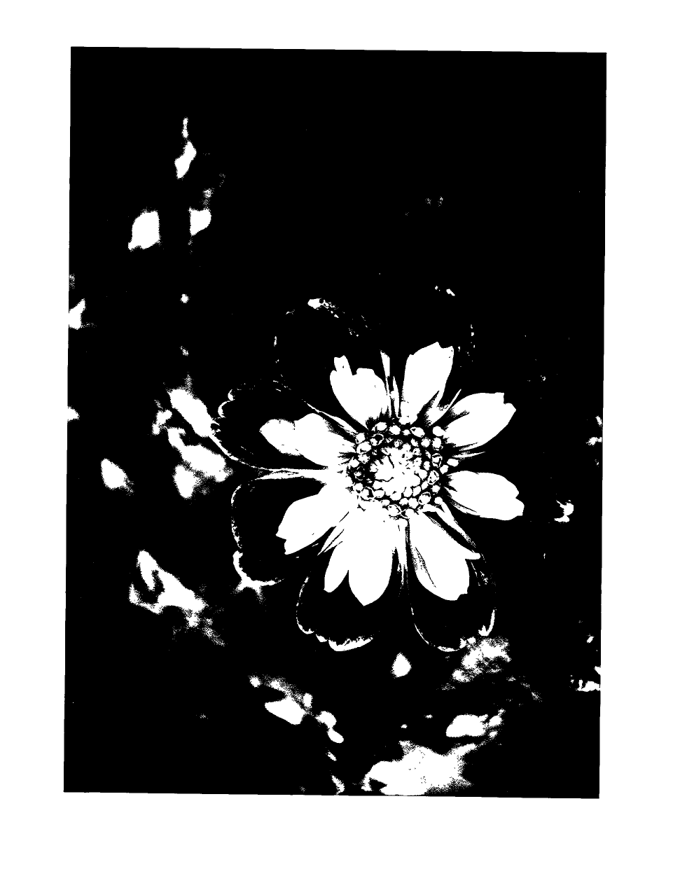

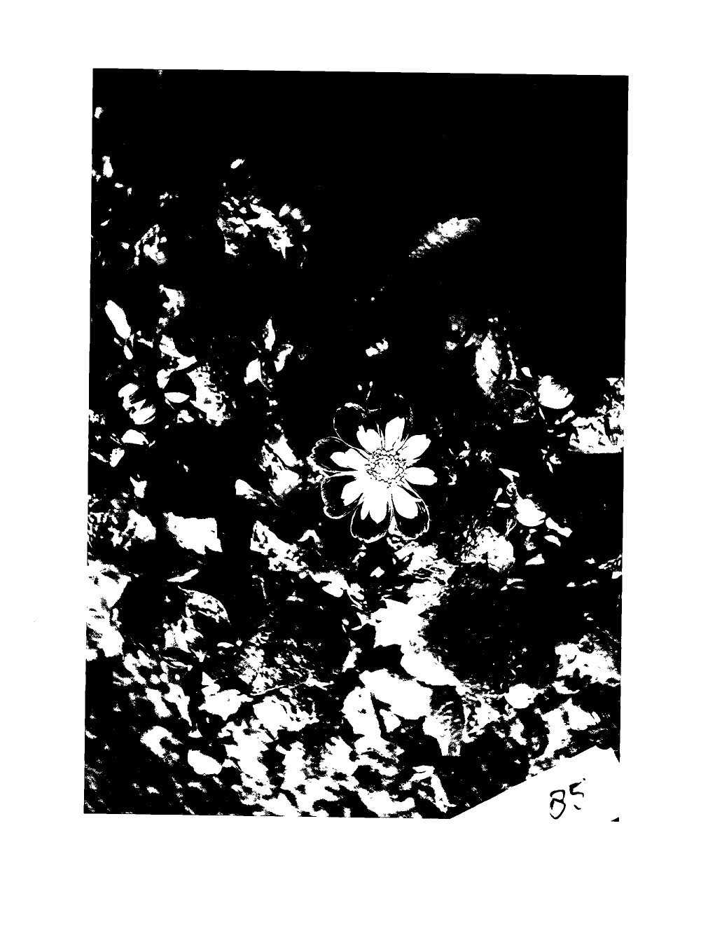

### BACKGROUND OF THE INVENTION

The present invention relates to a new and distinct variety of Dahlia hybrida (DAHLIA variabilis) known by the varietal denomination ‘HGD161-7’. The female parent of ‘HGD161-7’ is a selection from a proprietary breeding collection known as ‘DA-93’ and also the male parent is a selection from a proprietary breeding collection known as ‘DA-93’.

The new cultivar ‘HGD161-7’ is a product of a planned breeding program and was selected from the progeny of the stated cross made on September, 1998 by the Inventor at HEM GENETICS, Hemmerbuurt 98, 1607 LC Hem, the Netherlands. ‘HGD161-7’ was first asexually propagated on May, 1, 1999 by the Inventor at HEM GENETICS, Hemmerbuurt 98, 1607 LC Hem, the Netherlands.

The new variety is a dahlia plant useful for growing outdoors and under glass which produces attractive contrasting red and yellow-orange flowers. Among the traits possessed by the new variety which in combination distinguish it from other varieties known to the inventor are its attractive flowers with contrasting flower colors, vigorous growth, compact plants and fast response/early flowering.

Asexual reproduction by cuttings of the new variety in the Netherlands has demonstrated that the combination of characteristics as described herein for ‘HGD161-7’ are firmly fixed and are retained through successive generations of asexual reproduction.

### BRIEF DESCRIPTION OF THE INVENTION

‘HGD161-7’ is particularly characterized by the following:

1. Compact growing plants;

2. Fast flower responses;

3. Vigorous growth;

4. Abundance of flowers;

5. Shape of the flowers; and

6. Contrasting red/yellow-orange flower color.

Perhaps the closest comparison cultivar is ESTIVO. HGD161-7 is more compact and has a different flower color than ESTIVO. ‘HGD161-7’ has not been tested under all available environmental conditions and the phenotype may vary with variations in environmental conditions such as temperature, light intensity, day length and humidity, without a change in genotype of the plant.

### BRIEF DESCRIPTION OF THE DRAWINGS

The accompanying color photographic drawings show typical characteristics of ‘HGD161-7’. Sheet 1 is a top view of a plant. Sheet 2 is a close up of a flower.

### DETAILED BOTANICAL DESCRIPTION

The following observations, measurements and values describe plants grown under commercial conditions. All color references are measured against The Royal Horticultural Society (RHS) Colour Chart. Colors are approximate as color depends on horticultural practices such as light level and treatment rate, among others, without however any variance in genotype.

Parentage:

Female parent.—DA-93, a selection from a proprietary breeding collection.

Male parent.—DA-93, a selection from a proprietary breeding collection.

Plant:

Form.—globose, erected.

Height.—35 cm. Depending on cultivation conditions.

Branching characteristics.—normal.

Natural flowering season.—early May until mid November in moderate sea climate conditions at the northern hemisphere.

Hardiness.—good.

Disease resistance.—no data available.

Flower stem.—Length: 10-15 cm. depending on cultivation conditions Color: dark green RHS 144-A.

Foliage.—Quantity: normal Leaf Arrangement: in pairs Shape of leaf: oval, medium serrated Internode: 60 mm. depending on cultivation conditions Texture: Leaf upper side: smooth Leaf underside: smooth with veins Color: Leaf upper side: dark green RHS 137-B Leaf underside: dark green RHS 137-B Venation: irregular, staggering Petiole length: 45 mm. depending on cultivation conditions Stipules: none Stem color: dark green RHS 144-A.

Sepals (1).—Number: 5 Length: 15 mm. depending on cultivation conditions Width: 7 mm. depending on cultivation conditions Color: dark green RHS 146-A.

Second sepals (2).—Shape: Oval Color: yellow-green RHS 151-A.

Bud:

Form.—rounded.

Size.—10 mm. depending on cultivation conditions.

Color of ray florets.—yellow-green RHS 151-A.

Flower:

Inflorescence form.—half double.

Bloom form.—rounded.

Bloom diameter.—6 cm. depending on cultivation conditions.

Lasting quality.—not relevant.

Ray Florets.—Inner ray florets (3): Upper surface: oval; 25 mm; RHS 46-A Outer ray florets attachment (4): Upper surface: oval; 15 mm; RHS 1-C.

Discflorets (5).—Length: 10 mm. depending on cultivation conditions Shape: rounded to elongated Color: Yellow-orange RHS 21-A Fragrance: none Reproductive organs: Anthers: 2×5 mm. yellow-orange RHS 21-B Pollen: yellow-orange RHS 21-A Pistils: yellow RHS 12-A Character of ovaries: single seed, inferior.

I claim:

1. A new and distinct cultivar of Dahlia plant named ‘HGD161-7’, as described and illustrated.
